(19) United States
(12) Patent Application Publication
(10) Pub. No.: US 2011/0003823 A1
(43) Pub. Date: Jan. 6, 2011

(54) Compositions and methods for treatment of diseases and conditions associated with vasodilation and/or vascular leakage

(75) Inventor: Gerald Horn, Deerfield, IL (US)

Correspondence Address: WOOD, PHILLIPS, KATZ, CLARK & MORTIMER, 500 W. MADISON STREET, SUITE 3800, CHICAGO, IL, 60661, US

(73) Assignee: Alpha Synergy Development, Inc.

(21) Appl. No.: 12/800,942

(22) Filed: May 26, 2010

Related U.S. Application Data
(63) Continuation-in-part of application No. 12/798,929, filed on Apr. 14, 2010.
Application No. 12/798,929 is a continuation-in-part of application No. 12/460,970, filed on Jul. 27, 2009.
(60) Provisional application No. 61/137,714, filed on Aug. 1, 2008.
(60) Provisional application No. 61/192,777, filed on Sep. 22, 2008.
(60) Provisional application No. 61/203,120, filed on Dec. 18, 2008.
(60) Provisional application No. 61/207,481, filed on Feb. 12, 2009.
(60) Provisional application No. 61/287,518, filed on Dec. 17, 2009.

(51) Int. Cl. A61K 31/498; C07D 403/12; C07D 233/58; A61K 31/4174; A61K 31/4164; A61P 1/00; A61P 9/04

(52) U.S. Cl. 514/249; 544/353; 548/346.1; 514/396; 514/399

(57) ABSTRACT

The invention provides compositions and methods for treating diseases and conditions, including systemic diseases and conditions, through an intravenous administration of a selective α-2 adrenergic receptor agonists having a binding affinity of 300 fold or greater for α-2 over α-1 adrenergic receptors. The amounts of the selective α-2 adrenergic receptor agonists are substantially lower than the amounts normally used to cause sedation. The compositions preferably include dexmedetomidine.

### CROSS-REFERENCE TO RELATED APPLICATIONS

This application is a continuation-in-part of U.S. patent application Ser. No. 12/798,929, filed Apr. 14, 2010, which claims a priority of U.S. patent application Ser. No. 12/460,970, filed Jul. 27, 2009, which claims a priority of U.S. Provisional Application Ser. Nos. 61/137,714, filed on Aug. 1, 2008; 61/192,777, filed on Sep. 22, 2008; 61/203,120, filed on Dec. 18, 2008; and 61/207,481 filed on Feb. 12, 2009. This application also claims a priority of U.S. Provisional Application Ser. No. 61/287,518, filed on Dec. 17, 2009. The contents of the above-mentioned application are hereby incorporated by reference in their entirety.

### BACKGROUND OF THE INVENTION

Systemic diseases and disorders are often accompanied by vasodilation and/or vascular leakage. Pro-inflammatory cytokines are important molecules whose elevated levels trigger targeted vascular permeability increase with potentially severe chemical cascade events leading to inflammatory and other severe sequelae.

Vascular Endothelial Growth Factor (VEGF) is a primary ubiquitous cytokine, an important molecule produced by cells which stimulates the growth of new blood vessels. Among other functions, VEGF maintains vascular integrity and is therefore a critical regulator responsible for maintaining the proper functioning of the blood vessels, including vascular permeability.

Vascular permeability may be divided into three basic categories: basal (i.e., normal physiologic functions); acute (associated with various pulmonary pathologies); and chronic (associated with such conditions as, for example, tumor angiogenesis, chronic hypoxia, and others).

In many diseases and conditions (including but not limited to pulmonary and systemic diseases and conditions), acute vascular permeability is accompanied by an elevated level of VEGF. An elevated level of VEGF causes an inflammatory cascade, generally starting with vascular leakage and culminating with severe inflammation. Vascular leakage is primarily characterized by venular postcapillary leakage as a predominant component. This leakage involves several mechanisms and may include stripping of vascular endothelial cadherins (VE-cadherins) and formation of large endothelial gaps which leak plasma and proteins, including large proteins, such as fibrin as well as multiple pores with more serous exudate.

In associated pulmonary conditions the leaking plasma, proteins and remaining exudative debris leak into bronchi and smaller bronchioles. There, they are exposed to clotting factors which precipitate large fibrin clots that further reduce cillary mucous clearance and add mucous plugs. The gaps and pores attract neutrophils which tend to stick along the opening or are released into the intraluminal milleui. The cumulative result is inspissated (i.e., thickened/trapped) “secretions.” These accumulated secretions cause collapse of alveoli, further block mucous clearance, diminish alveolar gas exchange, attract water, solutes, and debris into the clots, and are very strong chemoattractants to additional neutrophils, promoting a strong inflammatory reaction as well as possibly releasing their own VEGF, cyclically exacerbating the pathophysiologic abnormal response, increasing fluid, inspissations, reduced alveolar gas exchange, and increasing the risk of infection and/or secondary infection due to stasis and reduced clearance of organic debris in the affected area(s). In addition, rebound vasodilation and hyperemia may occur, often accompanied by ischemia and additional inflammation.

VEGF is one of several pro-inflammatory cytokines capable of inducing potent microvessel permeability leakage, particularly at terminal arterioles, postcapillary venules and contiguous or near contiguous smaller venules. These include platelet activating factor (PAF), interleukins (e.g. IL-1), tumor necrosis factor (alpha-TNF), histamine, and serotonin; where the endothelial cell walls where large endothelial cell gaps and or increased pore formation is induced causing extensive microvascular leakage and a similar mechanism of inflammation induction. In the case of shock, particularly septic and/or anaphylactic shock, such extensive microvascular leakage quickly leads to loss of intravascular volume into interstitial space. Further, pro-inflammatory cytokines may induce increased gene expression of endothelins (particularly endothelin-1, but also endothelin 2,3), potent vasoconstrictor and smooth muscle constrictor peptides, implicated in a variety of conditions, including but not limited to: cerebrovascular accident morbidity, idiopathic pulmonary fibrosis, asthma, heart disease with atherosclerotic inflammatory and fibrotic changes, cancer, shock, renal failure, arthritis, and chronic pancreatitis. These endothelins are then released by a variety of cells including polymorphonuclear leukocytes, macrophages, and endothelial cells, and may be increased in a cascade of chemically induced sequelae following pro-inflammatory cytokine induced leakage and neutrophil extravasation and chemoattraction common to many systemic conditions.

Currently available means of treatment of the diseases associated with vascular permeability are inadequate. For example, treatment with vasopressors, such as vasopressin, dopamine, norephinephrine, and, to some degree, epinephrine (which has high α-1 selectivity, moderate α-2 selectivity, and is a moderate β-agonist), frequently leads to adrenal and/or renal failure from ischemic consequences of constriction of large vessels and microvessels. Further, currently available vasopressors may cause rebound vasodilation and rebound hyperemia, which significantly weaken whatever positive effect of vasopressors.

Thus, there is a need for new and/or improved means to reduce or otherwise prevent this pathologic sequence of events in these and similarly induced disease states without the adverse sequelae of alpha-1 ischemia attendant to use of vasopressors. There is also a need for new compositions and methods that would cause selective microvessel constriction and inhibit harmful effects of elevated VEGF on vascular permeability in various systemic diseases and/or disorders without increasing the risk of untoward imbalance and/or deterioration of essential vascular integrity and homeostasis.

### SUMMARY OF THE PRESENT INVENTION

The present invention provides compositions and methods to treat and/or prevent diseases and conditions associated with vasodilation and/or vascular leakage through an intravenous infusion and/or injection of a selective α-2 adrenergic receptor agonist having a binding affinity (Ki) of 300 fold or greater for α-2 over α-1 receptors at an amount which is substantially lower than that of said agonist normally used to cause sedation.

Preferably, the diseases and conditions are systemic diseases and conditions.

In some embodiments, the invention provides a method of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is continuously administered at a rate of between about 0.001 ng/min and about 70 ng/min for a 50 kilogram individual.

The rate of intravenous (IV) administration of between about 0.001 ng/min and about 70 ng/min for a 50 kg individual results in the administration of extremely low dose of the selective α-2 adrenergic receptor agonist.

In preferred embodiments, the rate of administration of the selective α-2 adrenergic receptor agonist is between about 0.01 ng/min and about 10 ng/min for a 50 kg individual.

In one embodiment, dexmedetomidine is administered at a rate of between 0.001 ng/min and about 70 ng/min, more preferably between 0.01 ng/min and about 10 ng/min, and still more preferably 0.05 and 0.5 ng/min for a 50 kg weight of a patient being treated). In prior art, dexmedetomidine was usually administered at about 160 ng/min and about 800 ng/min per 50 kg of weight of a patient for IV sedation.

For the purposes of the invention, dexmedetomidine is administered at a rate of well below 0.2 μg/kg/hr; e.g. 1.2×10⁻⁶ to 0.08 μg/kg/hr.

In another embodiment, brimonidine is administered at a rate of between about 0.01 ng/min and about 20 ng/min; preferably between about 0.05 ng/min and about 5 ng/min; and most preferably between about 0.1 ng/min and about 1 ng/min for a 50 kg individual.

While dexmedetomidine is known to have been used for an intravenous sedation, the current invention employs significantly lesser amounts of dexmedetomidine (or any other selective α-2 adrenergic receptor agonist), which are not enough to cause sedation or CNS cardiovascular effects, but are sufficient for the purposes of the invention: i.e. to cause relatively selective microvessel constriction and/or reverse rebound hyperemia.

Rebound hyperemia refers to induced vasodilation (instead of intended vasoconstriction) occurring, often with a lag time, after an application or, more typically, repeated applications of vasopressors (vasoconstrictors) and characterized by engorgement of blood vessels (such as those in the conjunctiva or nasal mucosa), increased capillary permeability and leakage, and, in some cases, inflammatory sequelae (medicamentosa, catecholamine refractory shock), frequently due to the use of an alpha-1 constricting drug repeatedly or at very high doses causing ischemia and induction of a pro-inflammatory cytokine cascade, and particularly with chronic use of a vasoconstricting drug.

In preferred embodiments of the invention, the selective α-2 adrenergic receptor agonists have Kᵢ for α-2 over α-1 receptors of 500:1 or greater. In more preferred embodiments of the invention, the selective α-2 adrenergic receptor agonists have Kᵢ for α-2 over α-1 receptors of 700:1 or greater. In more preferred embodiments of the invention, the selective α-2 adrenergic receptor agonists have Kᵢ for α-2 over α-1 receptors of 1000:1 or greater. In even more preferred embodiments of the invention, the selective α-2 adrenergic receptor agonists have Kᵢ for α-2 over α-1 receptors of 1500:1 or greater.

In preferred embodiments of the invention, the selective α-2 adrenergic receptor agonists have binding affinities (Kᵢ) of 100 fold or greater for α-2b and/or α-2c receptors over α-2a receptors.

In preferred embodiments of the invention, the selective α-2 adrenergic receptor agonist is selected from the group consisting of brimonidine, dexmedetomidine, fadolmidine, and mixtures of these compounds.

The most preferred selective α-2 adrenergic receptor agonist is dexmedetomidine.

In preferred embodiments, the compositions and methods of the invention may comprise potassium chloride and/or calcium chloride.

Preferably, the concentration of potassium chloride is between about 10 mM and 80 mM, most preferably about 20 mM to 40 mM, and the concentration of calcium chloride is between about 0.05 mM and about 2 mM, most preferably about 1 mM.

In preferred embodiments, a pH of the composition of the invention is between about 4.0 and about 6.5.

In some aspects, the compositions and methods of the invention further comprise other therapeutic agents, including bronchodilators and/or antibiotics.

In preferred embodiments, the bronchodilators may include, but are not limited to, selective and/or non-selective β-2 adrenergic receptor agonists, anticholinergics, and theophylline.

In some preferred embodiments, the invention provides a method of treating a systemic disease or condition associated with vasodilation and/or vascular leakage comprising intravenously continuously administering to a subject in need thereof dexmedetomidine, or a pharmaceutically acceptable salt thereof, at a rate of between about 0.001 ng/min to about 70 ng/min for a 50 kg individual (corresponding to between about 1.2×10⁻⁶ ng/kg/min to about 0.08 ng/kg/min).

In some embodiments, the invention provides a method of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is administered through an intramuscular injection or buccal administration at an amount of between about 0.0025 μg/kg to 0.5 μg/kg.

In preferred embodiments, the rate of administration of the selective α-2 adrenergic receptor agonist is between about 0.05 μg/kg to 1.25 μg/kg.

In some preferred embodiments, the invention provides a method of treating a systemic disease or condition associated with vasodilation and/or vascular leakage comprising intravenously administering to a subject in need thereof dexmedetomidine, or a pharmaceutically acceptable salt thereof, through an injection at an amount of between about 0.0025 μg/kg to 0.5 μg/kg.

In some embodiments, the invention provides a method of treating a systemic or gastrointestinal disease or condition associated with vasodilation and/or vascular leakage comprising administering to a subject in need thereof a highly selective alpha 2 agonist having a binding affinity of 300 fold or greater for α-2 over α-1 adrenergic receptors, or a pharmaceutically acceptable salt thereof, through nasal and/or oral administration at an amount which is 2 to 200,000 times lower than that of said agonist normally used to cause sedation

Without wishing to be bound to any particular theory, in preferred embodiments, the compositions and methods of the invention result in reduced vascular permeability believed to be caused by postcapillary venular constriction induced by the inventive compositions and methods. Thus, the compositions and methods of the invention reduce the large VEGF-induced postcapillary venular gaps and related vascular permeability increase, resulting in selective inhibition of the acute vascular permeability increase and related inflammatory and hypoxic sequelae caused by elevated levels of VEGF. This postcapillary venular constriction is believed to be increased in hypoxic conditions typical of pulmonary pathology associated with VEGF increase.

The compositions and methods of the present invention are believed to be capable of reducing vascular permeability, selectively inhibiting VEGF-induced postcapillary venular leakage, and/or selectively reducing spread of viral and/or bacterial pathogens.

Accordingly, in one embodiment, the invention provides methods of inducing a selective vasoconstriction of smaller blood vessels, such as microvessels, capillaries, and/or postcapillary venules relative to larger blood vessels, such as arteries and/or proximal arterioles. This selective vasoconstriction of smaller blood vessels allows for such effects while decreasing and/or eliminating ischemia risk. Unlike the present invention, α-1 agonists induce constriction of large and small vessels, for example causing constriction of the pulmonary artery, and/or end organ ischemia in treatment of septic and or anaphylactic shock with adrenal cortical insufficiency, renal failure, and other end organ failure high risk sequelae. Alpha-1 induced large vessel vasoconstriction may therefore cause additional ischemia, promote additional inflammation, and render desired vasoconstriction replaced by “rebound” vasoconstriction and related catecholamine resistant septic shock and/or anaphylactic shock that conventional pressors, with high alpha-1 activity, exacerbate. Therefore, α-1 agonists may considerably increase ischemia and secondarily inflammation. They are also direct agonist constrictors of bronchiole muscularis, which is equally or more damaging, since they cause direct bronchiole constriction, which is a highly deleterious and dangerous effect in respiratory compromised patients.

The inventive compositions and methods may also selectively inhibit VEGF-induced postcapillary venular leakage and/or reverse rebound hyperemia caused by α-1 agonists or other causes.

### BRIEF DESCRIPTION OF THE FIGURES

FIG. 1 is a graphical representation of dexmedetomidine infusion rate versus % postcapillary venular constriction

FIG. 2 is a graphical representation of effectiveness of the compositions of the present invention to selectively constrict the terminal arteriole, postcapillary venule, and/or adjacent small-moderate venules that are the primary sites targeted by pro-inflammatory cytokines for vascular leakage in a variety of disease conditions.

FIG. 3A is a baseline visual appearance of two eyes of a patient with an ocular condition of moderate hyperemia.

FIG. 3B depicts a visual appearance of the right eye of the patient after being treated with a prior art composition comprising VISINE Original® (tetrahydrozoline 0.05%) and the induction of rebound hyperemia, and the visual appearance of the left eye of the patient after being treated simultaneously with a composition of the present invention comprising brimonidine at 0.015%

FIG. 3C depicts a visual appearance of the right eye of the patient after then being treated with the novel composition of the present invention comprising brimonidine at 0.015%, reversing the VISINE Original® induced rebound hyperemia, and a visual appearance of the left eye of the patient after being treated simultaneously with an additional drop of the composition of the present invention comprising brimonidine at 0.015%.

### DETAILED DESCRIPTION OF THE INVENTION

### Definitions

The term “selective α-2 adrenergic receptor agonists” encompasses all α-2 adrenergic receptor agonists which have a binding affinity of 300 fold or greater for α-2 over α-1 adrenergic receptors. The term also encompasses pharmaceutically acceptable salts, esters, prodrugs, and other derivatives of selective α-2 adrenergic receptor agonists.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan™, and UK14304.

The term “dexmedetomidine” encompasses, without limitation, dexmedetomidine salts and other derivatives.

As used herein, the term “composition” is intended to encompass a product comprising the specified ingredients in the specified amounts, as well as any product which results, directly or indirectly, from a combination of the specified ingredients in the specified amounts.

The terms “treating” and “treatment” refer to reversing, alleviating, inhibiting, or slowing the progress of the disease, disorder, or condition to which such terms apply, or one or more symptoms of such disease, disorder, or condition.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

### Embodiments of the Invention

The present invention provides compositions and methods to treat and/or prevent diseases and conditions associated with vasodilation and/or vascular leakage through an intravenous infusion, nasal administration, oral, perioral and/or buccal administration, and/or injection, including intramuscular, of a selective α-2 adrenergic receptor agonist having a binding affinity (Ki) of 300 fold or greater for α-2 over α-1 receptors at an amount which is substantially lower than that of said agonist normally used to cause sedation.

In some embodiments, the invention provides a method of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is continuously administered at a rate of between about 0.001 ng/min and about 70 ng/min for a 50 kilogram individual.

The rate of intravenous (IV) administration of between about 0.001 ng/min and about 70 ng/min for a 50 kg individual results in the administration of extremely low dose of the selective α-2 adrenergic receptor agonist.

In preferred embodiments, the rate of administration of the selective α-2 adrenergic receptor agonist is between about 0.01 ng/min and about 10 ng/min for a 50 kg individual.

In one embodiment, dexmedetomidine is administered at a rate of between 0.001 ng/min and about 70 ng/min, more preferably between 0.01 ng/min and about 10 ng/min, and still more preferably 0.05 and 0.5 ng/min for a 50 kg weight of a patient being treated). In prior art, dexmedetomidine was usually administered at about 160 ng/min and about 800 ng/min per 50 kg of weight of a patient for IV sedation.

For the purposes of the invention, dexmedetomidine is administered at a rate of well below 0.2 μg/kg/hr; e.g. 1.2×10⁻⁶ to 0.08 μg/kg/hr.

In another embodiment, brimonidine is administered at a rate of between about 0.01 ng/min and about 20 ng/min; preferably between about 0.05 ng/min and about 5 ng/min; and most preferably between about 0.1 ng/min and about 1 ng/min for a 50 kg individual.

The compositions and methods of the present invention thus utilize extreme low doses of selective α-2 adrenergic receptor agonist, whereby these low doses cause peripheral microvessel constriction, with little or no effects on larger vessels and little or no hypertensive changes. As more fully explained below, microvessel constriction may be utilized for the treatment of wide variety of diseases.

When higher doses of selective α-2 adrenergic receptor agonists are intravenously administered (e.g., 0.2-1.0 μg/kg/hr), they cause sedative effects on the central nervous system (CNS), while the compositions and methods of the present invention produce little to none sedative effects.

When even higher doses of selective α-2 adrenergic receptor agonists are intravenously administered (e.g., greater than 1.0 μg/kg/hr), they cause peripheral vasoconstriction of larger vessels, which the present invention seeks to avoid.

Microvessel constriction resulting in reduced gap size and/or pore size restricts the release of plasma and exudative protein. Where alpha-2 receptors predominantly affect terminal arterioles and postcapillary venules, at sufficiently low concentrations of highly selective α-2 agonists as described above the result is highly selective interference of VEGF, endotoxin, and/or other pro-inflammatory cytokine induced microvessel leakage, as found in a substantial number of pathologic conditions, some of which are listed above.

The primary pro-inflammatory cytokine leakage occurs at terminal arterioles (about 10 microns) and postcapillary venules (about 10-20 microns). Secondary pro-inflammatory cytokine leakage occurs at medium or moderate size venules (about 20 to 40 microns) and tertiary pro-inflammatory cytokine leakage occurs at large venules (greater than about 40 microns)

Drug access to central nervous system (CNS) tissues is primarily limited by the blood brain barrier (BBB), and cerebrospinal fluid via choroid plexus and arachnoid membrane. The BBB is 95% microvasculature, and the plexus and arachnoid are similarly structured with a high preponderance of tight junction endothelial cells at the BBB and delivery across endothelial cells of the plexus and arachnoid membrane into cerebrospinal fluid (CSF). Microvessel vasoconstrictive effects limiting the diameter of pial and other relevant BBB and CSF microvessels by reducing the diameter of terminal arterioles, post capillary venules and the like can reduce the delivery of other drug into CNS in some instances, though highly lipophilic agonists themselves can cross the barrier relatively easily. Highly selective alpha-2 agonists at low concentrations as described to target primarily α-2 receptors can therefore reduce CNS crossover and still trigger microvessel cerebral constriction. In this manner, CNS neurons may be protected from the known adverse effects of chemotherapy, where in many cases neurons as part of the neurovascular unit are more sensitive to the chemotherapeutic drug than cancer cells and undergo apoptosis. The debilitating and often permanent CNS neuronal damage secondary to systemic chemotherapy may involve the neurovascular unit, where leakage of the microvessel associated with a neuronal cell and astroglial cell may cause permanent damage to the neuron.

Microvessel vasoconstriction and reduced release of neutrophils and related triggering of inflammatory cascade from large gaps/pores induced in endothelial cells during such induced microvessel leakage may help in treating diseases and conditions such as subarachnoid hemorrhage, and/or microvessel leakage via chemotherapy, endotoxin, VEGF elevation (as with altitude sickness) inflammation, alpha-1 agonist activity and attendant induced ischemia and inflammation (as may occur with vasopressor use, for example, in septic and/or anaphylactic shock), or induced microvessel permeability via pro-inflammatory cytokines, endotoxin, etc.

The compositions and methods of the present invention may thus provide a shield of microvessel constriction in the CNS and provide important protective advantages in chemotherapy.

Further, the present invention may directly reduce BBB and CSF chemotherapeutic levels and protect the neurovascular unit by reducing release of chemotherapeutic agents that cross the barrier from reaching the neuron. When CNS protection is desired, intravenous administration is a preferred route of delivery, while intranasal delivery can also be highly efficacious, particularly if atomized at particle size of 10-20 μM, with delivery reaching the upper nasal olfactory mucosa, just below the cribiform plate of the skull. Rapid CSF levels can be achieved without need to cross the blood brain barrier. This may for example provide a useful route of quick administration of the present invention to treat patients with altitude sickness or other causes of cerebral edema, acute brain dysfunction, or general CNS protection from chemotherapeutic cognitive, memory and related neuronal loss. For nasal mucosa absorption particle size of 10-20 μm is preferred, for more direct lung absorption via intranasal delivery bypassing the nasal cavity particle size of less than 2 μm is preferred.

Preferably, the diseases and conditions treatable with compositions and methods of the invention are systemic diseases and conditions with systemic manifestations of microvessel permeability induction. They include, but are not limited to, septic shock (endotoxin induced microvessel leakage), anaphylactic shock (platelet activating factor, histamine, serotonin and other proinflammatory cytokine induced leakage), systemic inflammatory response syndrome (SIRS), acute brain dysfunction, toxic shock syndrome ultra-fine carbon black inhalation, often associated acute lung injury (ALI), nonsmall cell lung cancer, idiopathic lung fibrosis, bronchiectatic form of cystic fibrosis, pneumonia bacterial including MRSA and strep and or viral, respiratory syncytial virus (RSV), eosinophilic pneumonia, whooping cough, asthma, status asthmaticus, lung transplantation, hantavirus pulmonary syndrome (HPS), hantavirus hemorrhagic fever with renal syndrome (HFRS), acute pancreatitis, acute prostatitis, acute, nephritis, and other acute inflammatory conditions to specific organs, inflammatory bowel disease including Crohn's disease and ulcerative colitis, Behcets disease, POEM constellation of diseases, eosinophilic meningitis, altitude sickness—particularly pulmonary and or cerebral edema, atherosclerotic heart disease, cerebrovascular accidents, chronic pancreatitis, among other conditions.

Often, although not always, systemic diseases and conditions involve inflammation. VEGF elevation, platelet activating factor (PAF), histamine, serotonin, and/or other proinflammatory cytokines. The resulting pathophysiology is highly targeted to postcapillary venular and/or adjacent venular leakage where α-2 agonist receptor endothelial cell population is highly distributed in humans and such microvessels effectively constricted.

Systemic conditions related to arteriovascular anomalies that may be treated with the present invention may include Hereditary Hemorrhagic Telangiectasia (HHT) in which such malformations are present throughout the body, but where epistaxis is particularly prevalent and difficult to treat, as well as other causes of epistaxis.

Thus, the compositions and methods of the present invention utilize extreme low doses of highly selective α-2 agonists (below clinically used doses of α-2 agonists) which allow for an extremely clinically important predominant microvessel constriction without recruitment of larger vessel constriction.

Because a large variety of diseases involve dilated microvessels, the compositions and methods of the invention, can treat a large range of pathophysiology, including but not limited to, systemic diseases, vascular permeability induction (by proinflammatory cytokines or other causes); inflammation (causes microvessel leakage); tissue or interstitial edema (result of such leakage); and cosmetic (effect of microvessel vasoconstriction for example to whiten the conjunctiva of they eye). The compositions and methods may also be used to shield normal tissue with normal microvessel constriction from toxic drug effects via reduced permeability vs. targeted tissue with abnormal vasculature where such microvessel constriction is less likely (e.g., tumors); or to protect the CNS.

In addition, the compositions and methods of the present invention may reduce the risk of bradycardia and/or hypotensive effect. Both bradycardia and hypotension are mediated via α-2 receptors and presynaptic norepinephrine release within receptors of the brain.

The invention provides compositions and methods that can be used for the treatment of fluid extravasation/loss in burn patients.

The present invention also provides combinations of highly selective α-2 adrenergic receptor agonists and anticholinergics. These combinations may provide further protection from bradycardia and related undesirable cardiovascular side effects. These combinations may also be used in the treatment of asthma, diseases of the GI tract (including irritable bowel syndrome (IBS)), as a sleeping pill, and in other diseases and/or conditions.

In a preferred embodiment, an anticholinergic is glycopyrrolate.

In a preferred embodiment, the invention provides a composition comprising dexmedetomidine and glycopyrrolate, wherein the composition is formulated for an intravenous (IV) administration, and wherein the amount of glycopyrrolate is between 0.01 and 0.2 mg, and more preferably between 0.05 and 0.1 mg.

In another preferred embodiment, the invention provides a composition comprising dexmedetomidine and glycopyrrolate, wherein the composition is formulated for an oral administration, and wherein the amount of glycopyrrolate is between 0.25 and 20 mg bid-tid (i.e. twice daily-three times daily), and more preferably between 0.5 and 1 mg bid-tid.

### Selective α-2 Adrenergic Receptor Agonists Suitable for the Purposes of the Invention

The selective α-2 adrenergic receptor agonists suitable for the purposes of the invention have binding affinities (Kᵢ) for α-2 over α-1 receptors of 300:1 or greater. In preferred embodiments of the invention, the selective α-2 adrenergic receptor agonists have Kᵢ for α-2 over α-1 receptors of 500:1 or greater. In more preferred embodiments of the invention, the selective α-2 adrenergic receptor agonists have Kᵢ for α-2 over α-1 receptors of 700:1 or greater. In more preferred embodiments of the invention, the selective α-2 adrenergic receptor agonists have Kᵢ for α-2 over α-1 receptors of 1000:1 or greater. In even more preferred embodiments of the invention, the selective α-2 adrenergic receptor agonists have Kᵢ for α-2 over α-1 receptors of 1500:1 or greater.

It is well within a skill in the art to design an assay to determine α-2/α-1 functional selectivity. As non-limiting examples, potency, activity or EC₅₀ at an α-2A receptor can be determined by assaying for inhibition of adenylate cyclase activity. Furthermore, inhibition of adenylate cyclase activity can be assayed, without limitation, in PC12 cells stably expressing an α-2A receptor such as a human α-2A receptor. As further non-limiting examples, potency, activity or EC₅₀ at an α-1A receptor can be determined by assaying for intracellular calcium. Intracellular calcium can be assayed, without limitation, in HEK293 cells stably expressing an α-1A receptor, such as a bovine α-1A receptor.

The particularly preferred adrenergic receptor agonists for the purposes of the present invention have higher selectivity for α-2B and/or α-2C receptors, as compared to α-2A receptors within the lung. In preferred embodiments of the invention, the selective α-2 adrenergic receptor agonists have binding affinities (Kᵢ) of 100 fold or greater for α-2b and/or α-2c receptors over α-2a receptors. While not wishing to be bound to any specific theory, it is believed that α-2b receptors have the predominant peripheral vascular and microvascular vasoconstrictive role in arterioles and venules, particularly terminal arterioles, postcapillary venules, and adjacent venules where most microvessel leakage occurs. At the same time, α-2a receptors are predominantly found in the central nervous system, and therefore, α-2a specific agonists have a lesser role in causing direct vascular constriction and reduction of vascular permeability but in many conditions may provide secondary benefits of sedation, reduced anxiety, and bronchiole dilation.

In addition to the α-2 and preferential α-2b agonist-induced microvessel terminal arteriolar and postcapillary venular constriction, a further advantage of the inventive compositions and methods may be activation of central nervous system (CNS) α-2a receptors. Activation of CNS α-2a receptors has been shown to have sedative effects, which may be beneficial in some diseases and conditions being treated with the compositions and methods of the present invention. For example, in bronchial constriction, anxiety and emotional stress are often contributing factors. CNS α-2a receptors are also thought to be involved in a mechanism inducing bronchiole dilation.

Compositions and methods of the inventions encompass all isomeric forms of the described α-2 adrenergic receptor agonists, their racemic mixtures, enol forms, solvated and unsolvated forms, analogs, prodrugs, derivatives, including but not limited to esters and ethers, and pharmaceutically acceptable salts, including acid addition salts. Examples of suitable acids for salt formation are hydrochloric, sulfuric, phosphoric, acetic, citric, oxalic, malonic, salicylic, malic, furmaric, succinic, ascorbic, maleic, methanesulfonic, tartaric, and other mineral carboxylic acids well known to those in the art. The salts may be prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66: 1-19 (1977) which is incorporated herein by reference).

As long as a particular isomer, salt, analog, prodrug or other derivative of a selective α-2 adrenergic receptor agonist functions as a selective α-2 agonist, it may be used for the purposes of the present invention.

When choosing a particular α-2 adrenergic receptor agonist, one may take into account various considerations including any possible side effects and other systemic reactions.

In select circumstances, it may be preferable for the active agent of the present invention to penetrate parenchymal cell membranes, in which case a higher pH, including pH of greater than 7 may be desired. In this event, solubility may be reduced and require anionic components to stabilize. Such anionic components may include peroxide and/or other solubility enhancers and/or preservatives.

In preferred embodiments of the invention, the selective α-2 adrenergic receptor is dexmedetomidine or its salt.

### Compositions and Methods of the Invention

In one embodiment, the invention provides a composition comprising a selective α-2 adrenergic receptor agonist having a binding affinity of 300 fold or greater for α-2 over α-1 adrenergic receptors, or a pharmaceutically acceptable salt thereof, wherein said composition is formulated for the treatment of a disease or condition associated with vasodilation and/or vascular leakage through an intravenous infusion and/or injection of said α-2 adrenergic receptor agonist at an amount which is substantially lower than that of said agonist normally used to cause sedation.

In other embodiments, the compositions of the invention may be delivered through other administration routes, including but not limited to nasal, oral, perioral and/or buccal administration.

In one embodiment, the selective α-2 adrenergic receptor is selected from the group consisting of brimonidine, dexmedetomidine, fadolmidine, and mixtures of these compounds.

In a preferred embodiment, the composition comprises dexmedetomidine.

In a more preferred embodiment, a pH of the composition comprising the selective α-2 adrenergic receptor agonist is between about 4.0 and about 6.5.

In another preferred embodiment, the compositions of the present invention further include potassium (i.e., K⁺). The term “potassium” includes, but is not limited to, potassium salt. In a preferred embodiment, potassium is in the form of potassium chloride (KCl) and its concentration is between about 10 mM and 60 mM, preferably between about 20 mM and about 40 mM most preferably about 40 mM.

In another preferred embodiment, the compositions of the present invention further include calcium (i.e., Ca²⁺). The term “calcium” includes, but is not limited to, calcium salt. In a preferred embodiment, calcium is in the form of calcium chloride (CaCl₂) and its concentration is between about 0.05 mM and 4 mM, more preferably between about 0.5 mM and 4 mM most preferably about 1.5 mM.

pH may range from 4.0 to 8.0, where the low concentration reduces stability concerns at pH above 7.0, and where anionic components may include peroxide preservatives to further stabilize. In preferred embodiments, a pH of the composition of the invention is between about 4.0 and about 6.5.

In some aspects, the compositions and methods of the invention further comprise other therapeutic agents, including bronchodilators and/or antibiotics.

In preferred embodiments, the bronchodilators may include, but are not limited to, β-2 adrenergic receptor agonists, anticholinergics, and theophylline.

The compositions of the present invention are preferably formulated for a mammal, and more preferably, for a human.

It is believed to be within a skill in the art to formulate the compositions for an intravenous (IV) administration or injection.

The invention also provides methods of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is continuously administered at a rate of between about 0.001 ng/kg/min and about 70 ng/kg/min.

In preferred embodiments, the rate of administration of the selective α-2 adrenergic receptor agonist is between about 1.2×10⁻⁶ ng/kg/hr and 0.08 ng/kg/hr.

In one embodiment, dexmedetomidine is administered at a rate of between 0.001 ng/min and about 70 ng/min, more preferably between 0.1 ng/min and about 1 ng/min (whereas in prior art dexmeditomidine was administered at about 160 ng/min and about 800 ng/min per 50 kg of weight of a patient for IV sedation).

In another embodiment, brimonidine is administered at a rate of between about 0.01 ng/min and about 20 ng/min; preferably between about 0.05 ng/min and about 5 ng/min; and most preferably between about 0.1 ng/min and about 1 ng/min (per 50 kg of weight of a patient for IV sedation).

In some preferred embodiments, the invention provides a method of treating a systemic disease or condition associated with vasodilation and/or vascular leakage comprising intravenously continuously administering to a subject in need thereof dexmedetomidine, or a pharmaceutically acceptable salt thereof, at a rate of between about 2.4×10⁻⁸ ng/kg/min to about 0.5 ng/kg/min, more preferably 2×10⁻⁴ ng/kg/min to 2×10⁻² ng/kg/min.

In some embodiments, the invention provides a method of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is administered through an injection at an amount of between about 0.0025 μg/kg and about 1.0 μg/kg.

In preferred embodiments, the rate of administration of the selective α-2 adrenergic receptor agonist is between about 0.05 μg/kg and about 0.1 μmole/kg.

In some preferred embodiments, the invention provides a method of treating a systemic disease or condition associated with vasodilation and/or vascular leakage comprising intravenously administering to a subject in need thereof dexmedetomidine, or a pharmaceutically acceptable salt thereof, through an injection at an amount of between about 0.005 μg/kg and about 0.25 μg/kg.

In preferred embodiments, the compositions and methods of the invention cause postcapillary venular constriction thus counteracting the clinically damaging increase in acute vascular permeability caused by elevated levels of VEGF.

In some embodiments, the compositions and methods of the invention selectively inhibit VEGF-induced postcapillary venular leakage.

In some embodiments, the compositions and methods of the invention selectively reduce the spread of viral and/or bacterial pathogens.

In some embodiments, the invention provides methods of inducing a selective vasoconstriction of smaller blood vessels, such as microvessels, capillaries, and/or postcapillary venules relative to larger blood vessels, such as arteries and/or arterioles. This selective vasoconstriction of smaller blood vessels allows decreasing and/or eliminating ischemia.

The compositions of the invention may also comprise a solubility stabilizer which preferably contains an anionic component, such as peroxide class preservatives. The solubility stabilizer allows one to achieve greater penetration of lipophilic membranes. In a preferred embodiment, the solubility stabilizer comprises a stabilized oxychloro complex, chlorite, and sodium perborate.

The compositions of the present invention may comprise nitrous oxide inhibitors. In a preferred embodiment, the nitrous oxide inhibitors are selected from the group consisting of L-NAME (L-N^{G}-Nitroarginine methyl ester), L-NIL (N6-(1-Iminoethyl)-L-lysine dihydrochloride), L-NIO (N5-(1-Iminoethyl)-L-ornithine dihydrochloride), and L-canavine, or combinations thereof. Preferably, concentration of the nitrous oxide inhibitors is between about 0.005% and about 0.5% weight by volume and/or between 0.1 μM and 20 μM, more preferably between 1 μM and 10 μM, and even more preferably between 2 μm and 6 μM.

The compositions may also include non-steroidal anti-inflammatory drugs, for example, indomethacin. In one embodiment, the concentration of indomethacin is between 0.1 and 10 μM, more preferably between 0.5 and 5 μM, and more preferably between 1-2 μM.

The invention also contemplates topical compositions which include, but are not limited to, gels and creams. They may also include additional non-therapeutic components, which include, but are not limited to, preservatives, delivery vehicles, tonicity adjustors, buffers, pH adjustors, antioxidants, tenacity adjusting agents, viscosity adjusting agents, and water.

Preservatives include, but are not limited to, benzalkonium chloride, chlorobutanol, thimerosal, phenylmercuric acetate, or phenylmercuric nitrate.

Delivery vehicles include, but are not limited to, polyvinyl alcohol, povidone, hydroxypropyl methyl cellulose, poloxamers, carboxymethyl cellulose, hydroxyethyl cellulose and purified water. It is also possible to use a physiological saline solution as a major vehicle.

Tonicity adjustors include, but are not limited to, a salt such as sodium chloride, potassium chloride, mannitol or glycerin, or another pharmaceutically or ophthalmically acceptable tonicity adjustor.

Tenacity adjusting agents include, but are not limited to, glycerin.

Viscosity adjusting agents include, but are not limited to, hypromellose (HPMC).

Buffers and pH adjustors include, but are not limited to, acetate buffers, citrate buffers, phosphate buffers and borate buffers, such as boric acid. It is understood that various acids or bases can be used to adjust the pH of the composition as needed. pH adjusting agents include, but are not limited to, sodium hydroxide and hydrochloric acid.

Antioxidants include, but are not limited to, sodium metabisulfite, sodium thiosulfate, acetylcysteine, butylated hydroxyanisole and butylated hydroxytoluene.

To make the topical compositions of the present invention, one can simply dilute, using methods known in the art, more concentrated solutions of selective α-2 agonists. The precise method of carrying out the dilutions is not critical. Any commonly used diluents, including preservatives described above in the application, suitable for topical solutions can be used.

Diseases and Conditions to be Treated with Compositions and Methods of the Invention

The diseases and conditions that can be treated or prevented using the compositions and methods of the invention include any diseases and conditions associated with vasodilation and/or vascular leakage. Microvessel leakage and inflammation are found in many diseases which are therefore potentially treatable with the compositions and methods of the present invention.

The compositions and methods of the invention may be used as either the primary or adjunctive treatment or both.

The diseases and conditions include but are not limited to, systemic diseases and conditions.

Systemic diseases and conditions include, but are not limited to, septic shock, systemic inflammatory response syndrome (SIRS), acute lung injury (ALI), toxic shock syndrome, acute pancreatitis, Crohn's disease and others. Often, although not always, systemic diseases and conditions involve inflammation.

The present invention is more fully demonstrated by reference to the accompanying drawings.

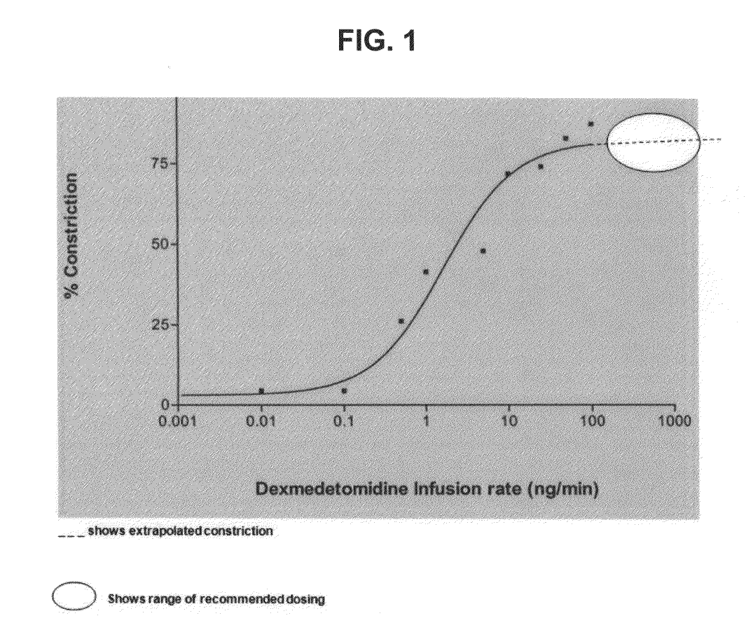

FIG. 1 is a graphical representation of dexmedetomidine infusion rate versus % postcapillary venular constriction. FIG. 1 demonstrates that the range of the infusion rate suitable for the purposes of the invention is generally between 0.01 ng/min and 10 ng/min. At a rate significantly above 10 ng/min, the undesirable stimulation of α-1 receptors becomes significant. For systemic conditions, it is desirable to achieve all vasoconstriction (including terminal arteriolar, postcapillary venular and some larger venular constriction) without significant α-1 agonist activity may be useful.

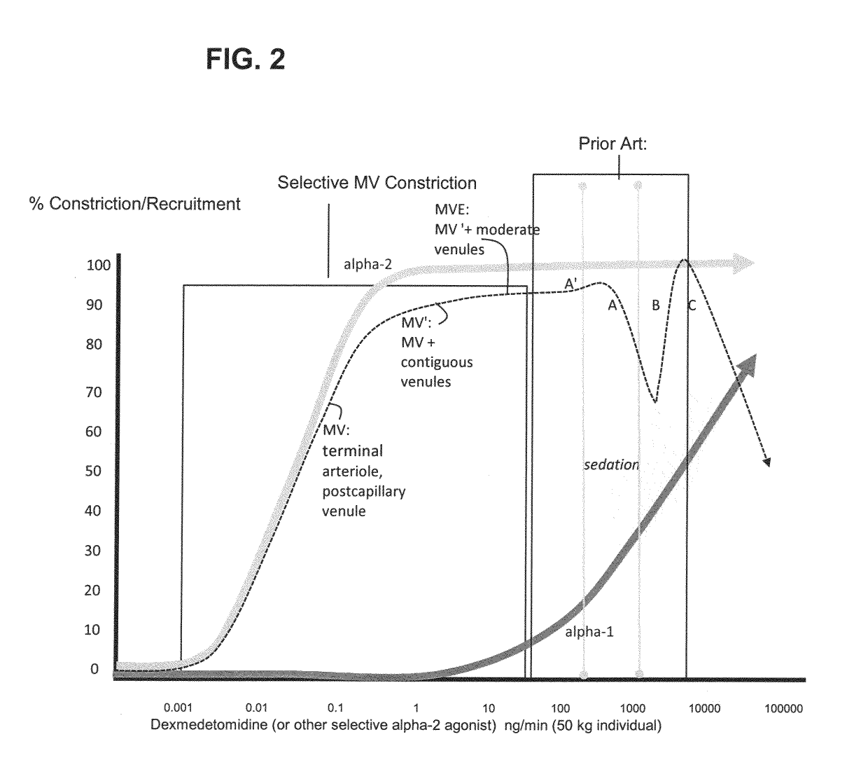

FIG. 2 is a graphical representation of effectiveness of the compositions of the present invention. As the chart illustrates, the invention provides extreme low doses of dexmedetomidine (or other selective α-2 agonists) for constriction of terminal arterioles and postcapillary venules and for reversing rebound hyperemia. As FIG. 2 demonstrates, dexmedetomidine may be administered at the rate of 0.001 to 70 ng/min, preferably 0.01 to 10 ng/min; and most preferably 0.05 to 5 ng/min (per 50 kg weight of a patient). These values correspond, accordingly, to 1.2×10⁻⁶ to 0.08; 1.2×10⁻⁵ to 0.012 and 7.2×10⁻⁵ to 0.00072 μg/kg/hr.

At the range of 0.001 to 70 ng/min, one may observe constriction of terminal arterioles; postcapillary venules; adjacent venules; and moderate size venules.

At the range of 0.01 to 10 ng/min, one may observe constriction of terminal arterioles; postcapillary venules; and adjacent venules.

At the range of 0.05 to 0.5 ng/min, one may observe constriction of terminal arterioles and postcapillary venules.

In FIG. 2, the following abbreviations are used:
MV means “microvessel contraction” (terminal arterioles and postcapillary venules); lumen size about 10-20 microns;
MV′ means MV plus contiguous vessels (lumen size about 20-30 microns);
MVE means MV′ plus moderate size venules (lumen size about 30-40 microns).

At the amounts significantly higher than about 70 ng/min per 50 kg weight, the activation of α-1 receptors becomes significant.

Concentration in the A′ area is such that there is significant alpha-1 recruitment for α-2 potentiation of α-1 effects.

Concentration in the A area is sufficient for sedation and CNS-induced vasodilation.

Concentration in the B area is sufficient for sedation large α-1 recruitment population and large artery (lumen size 100-1000 microns) vasoconstriction.

Concentration in the C area is sufficient for α-1 large vessel constriction induced ischemia, inflammation, vascular leakage and rebound hyperemia.

The administration rates significantly higher than 100 ng/min result in sedation, CNS induced cardiovascular changes and activation of α-1 receptors

The following Examples are provided solely for illustrative purposes and is not meant to limit the invention in any way.

### Example 1

### Reversing Alpha-1 Agonist Induced Ischemia and Rebound Hyperemia

This experiment demonstrates that the compositions and methods of the present invention are able to reverse α-1 agonist induced ischemia and rebound hyperemia. The Example is best illustrated through FIGS. 4A-4C.

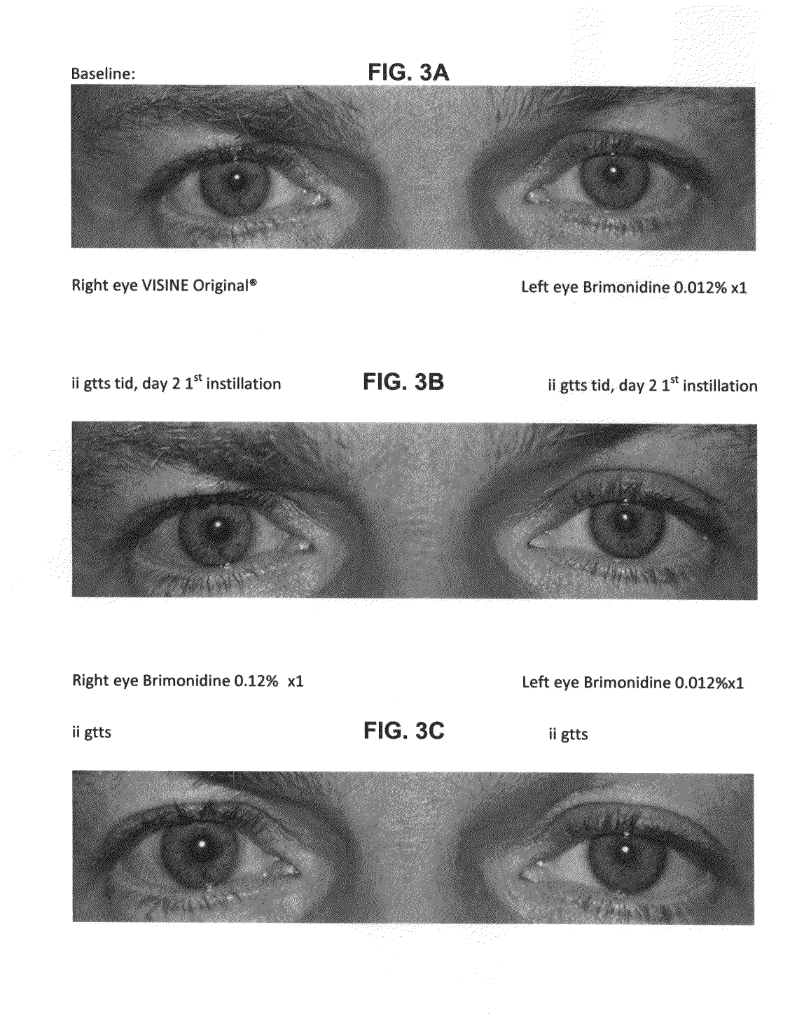

FIG. 3A is a baseline visual appearance of two eyes of a patient with an ocular condition.

FIG. 3B depicts a visual appearance of the right eye of the patient after being treated with a prior art composition comprising VISINE Original® (Johnson & Johnson's registered trademark; active ingredient: tetrahydrozoline HCL 0.05%). The treatment induced rebound hyperemia in the right eye. The left eye of the patient after being treated simultaneously with a composition of the present invention comprising brimonidine at 0.015% is free of hyperemia.

FIG. 3C depicts a visual appearance of the right eye of the patient after then being treated with the composition of the present invention comprising brimonidine at 0.015%, reversing the VISINE Original® induced rebound hyperemia, and a visual appearance of the left eye of the patient after being treated simultaneously with an additional drop of the composition of the present invention comprising brimonidine at 0.015%.

This experiment demonstrates that the compositions of the present invention are able to reduce inflammation, constrict microvessels, and cause reversal of rebound hyperemia. While the experiment explored the effect of extremely low dose brimonidine on a rebound hyperemia in the eyes, it is believed that similar effects will be achieved for treating non-ophthalmic diseases and conditions, as described in the present invention.

### Example 2

### Prophetic

### Effect of Brimonidine and Dexmedetomidine on Inhibition of VEGF Inflammatory Cascade

The purpose of this experiment is to test the effect of administering aerosolized brimonidine and dexmedetomidine on pulmonary function and vascular leakage via lung weight measurement in acute respiratory viral infection.

Study Design

A parallel group design of five groups of eight rats each: virus/saline, virus/brimonidine, virus/dexmedetomidine, sham/saline, sham/brimonidine. Treatments are twice daily, beginning one day post inoculation, and ending the morning of terminal studies on day 4, 5 or 6 post inoculation.

Treatments
1) Brimonidine tartrate 0.05% aerosol, generated with ultrasonic nebulizer (12 ml solution loaded into nebulizer for each treatment), delivered into a holding chamber, and breathed spontaneously by awake rats for 5 minutes twice daily (0800 and 1800 hrs), beginning eight hours after viral inoculation.
2) Dexmedetomidine HCl 0.05% aerosol, generated with ultrasonic nebulizer (12 ml solution loaded into nebulizer for each treatment), delivered into a holding chamber, and breathed spontaneously by awake rats for 5 minutes twice daily (0800 and 1800 hrs), beginning one day after viral inoculation.
3) Control treatment: pH-matched saline aerosol.

A 5-minute exposure is recommended due to the lag time of filling the exposure box with aerosol after the rats have been loaded into the box.

### Viral Infection

Rats will be inoculated with Parainfluenza type 1 (Sendai) virus via aerosol exposure, and housed in isolation cubicles. Control groups will be sham-inoculated with virus-free vehicle, and housed in an identical manner.

### Assessment

daily body weights;
lung function: oxygenation on room air (pulse oximetry); lung mechanics (pressure-volume curve, quasistatic elastance, dynamic elastance); airflow resistance (Newtonian resistance, respiratory system resistance, tissue damping);
lung inflammation: right lung bronchoalveolar lavage, with total leukocyte and differential leukocyte counts;
pulmonary transudate & exudates: left lung wet/dry weight ratio determined for 6 rats in each group;
formalin-fixed, paraffin-imbedded left lungs from 2 rats in each group; mid-sagittal thin sections prepared with H&E stain.

What is claimed is:

1. A composition comprising a selective α-2 adrenergic receptor agonist having a binding affinity of 300 fold or greater for α-2 over α-1 adrenergic receptors, or a pharmaceutically acceptable salt thereof, wherein said composition is formulated for the treatment of a disease or condition associated with vasodilation and/or vascular leakage through an intravenous infusion and/or injection of said α-2 adrenergic receptor agonist, at an amount which is substantially lower than that of said agonist normally used to cause sedation.

2. The composition of claim 1, wherein said disease or condition is a systemic disease or condition.

3. The composition of claim 1, wherein said selective α-2 adrenergic receptor agonist has a binding affinity of 700 fold or greater for α-2 over α-1 adrenergic receptors.

4. The composition of claim 1, wherein said selective α-2 adrenergic receptor agonist has a binding affinity of 1000 fold or greater for α-2 over α-1 adrenergic receptors.

5. The composition of claim 1, wherein said selective α-2 adrenergic receptor has a binding affinity of 100 fold or greater for α-2b and/or α-2c receptors over α-2a adrenergic receptors

6. The composition of claim 1, wherein said selective α-2 adrenergic receptor agonist is selected from the group consisting of brimonidine, dexmedetomidine, and mixtures of these compounds.

7. The composition of claim 1, wherein said composition further comprises potassium chloride.

8. The composition of claim 1, wherein said composition further comprises calcium chloride.

9. A method of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is continuously administered at a rate of between about 0.001 ng/min and about 100 ng/min for a 50 kg individual.

10. The method of claim 9, wherein said selective α-2 adrenergic receptor agonist is administered at a rate of between about 0.05 ng/min to about 10 ng/min.

11. The method of claim 9, wherein said selective α-2 adrenergic receptor agonist is dexmedetomidine.

12. The method of claim 9, wherein said selective α-2 adrenergic receptor agonist is brimonidine.

13. A method of treating a systemic disease or condition associated with vasodilation and/or vascular leakage comprising intravenously continuously administering to a subject in need thereof dexmedetomidine, or a pharmaceutically acceptable salt thereof, at a rate of between about 0.001 ng/min and about 100 ng min.

14. A method of treating a systemic disease or condition associated with vasodilation and/or vascular leakage comprising intravenously administering to a subject in need thereof a selective α-2 adrenergic receptor agonist having a binding affinity of 300 fold or greater for α-2 over α-1 adrenergic receptors, or a pharmaceutically acceptable salt thereof, wherein said selective α-2 adrenergic receptor agonist is administered through an injection at an amount of between about 0.0025 μg/kg and 1.25 μg/kg.

15. The method of claim 14, wherein said selective α-2 adrenergic receptor agonist is administered through an injection at an amount of between about 0.05 μg/kg and about 0.1 μg/kg.

16. The method of claim 15, wherein said selective α-2 adrenergic receptor agonist is dexmedetomidine.

17. The method of claim 15, wherein said selective α-2 adrenergic receptor agonist is brimonidine.

18. A method of treating a systemic disease or condition associated with vasodilation and/or vascular leakage comprising intravenously administering to a subject in need thereof dexmedetomidine, or a pharmaceutically acceptable salt thereof, through an injection at an amount of between about 0.005 μg/kg and about 0.25 μg/kg.

19. The method of claim 18 where said systemic disease or condition is selected from the group consisting of septic shock, anaphylactic shock, toxic shock syndrome, hyperlipidemia, atherosclerotic heart disease, cerebrovascular accidents, and systemic and CNS toxicity of chemotherapy.

20. A method of treating a systemic or gastrointestinal disease or condition associated with vasodilation and/or vascular leakage comprising administering to a subject in need thereof a selective alpha 2 agonist having a binding affinity of 300 fold or greater for α-2 over α-1 adrenergic receptors, or a pharmaceutically acceptable salt thereof, through nasal and/or oral administration at an amount which is 2 to 5,000 times lower than that of said agonist normally used to cause sedation.

21. The method of claim 20 where said selective alpha 2 agonist has reduced blood brain barrier permeability than dexmedetomidine and/or brimonidine.

22. The method of claim 21 where said alpha 2 agonist is fadolmidine.
